(19) United States
(12) Patent Application Publication
(10) Pub. No.: US 2011/0123610 A1
(43) Pub. Date: May 26, 2011

(54) EXTENDED RELEASE COMPOSITIONS CONTAINING TOLTERODINE AND PROCESS FOR PREPARING THE SAME

(75) Inventors: Rajesh KSHIRSAGAR, Bangalore (IN); Sachin MUNDADE, Bangalore (IN); Ganesh SHINDE, Bangalore (IN); Pravin KAMBLE, Bangalore (IN); Sandip SONAWANE, Bangalore (IN); SM MUDDA, Bangalore (IN)

(73) Assignee: MICRO LABS LIMITED, Bangalore (IN)

(21) Appl. No.: 12/951,965

(22) Filed: Nov. 22, 2010

(30) Foreign Application Priority Data
Nov. 23, 2009 (IN) 2880/CHE/2009

(51) Int. Cl. A61K 9/52; A61K 31/135; A61P 13/00

(52) U.S. Cl. 424/456; 514/648

(57) ABSTRACT

The present invention is directed to an extended release pharmaceutical composition such as tablets and capsules, and in particular to a matrix tablet composition comprising a therapeutically effective quantity of Tolterodine or pharmaceutical acceptable salts thereof incorporated in a hydrophobic matrix comprising water insoluble polymer and wax and a method for the preparation thereof.

### TECHNICAL FIELD OF THE INVENTION

The present invention relates to an extended release pharmaceutical composition such as tablets and capsules, and in particular to a matrix tablet composition for oral administration comprising a therapeutically effective quantity of Tolterodine or pharmaceutical acceptable salts thereof incorporated in a hydrophobic matrix comprising water insoluble polymer and wax and a method for the preparation thereof.

### BACKGROUND OF THE INVENTION

Urinary incontinence is the involuntary excretion of urine from one's body. It is assumed that unstable or overactive bladder is caused by uncontrolled contractions of the bundles of smooth muscle fibers, forming the muscular coat of the urinary bladder, during its filling phase. The pharmacological treatment in such cases is the administration of muscarinic receptor antagonists such as Oxybutynin and Tolterodine.

Tolterodine is the (R)—N,N-diisopropyl-3-(2-hydroxy-5-methylphenyl)-3-phenylpropylamine and is an antimuscarinic drug that is used to treat urinary incontinence and other symptoms of unstable or overactive urinary bladder. It acts on M2 and M3 subtypes of muscarinic receptors whereas most antimuscarinic agents only act on M3 receptors. Tolterodine targets the bladder more than other areas of the body thus lower dose needs to be given daily (due to efficient targeting) and so causing fewer side effects.

Various methods are already known for the industrial preparation of extended release oral dosage forms comprising an antimuscarinic agent, and in particular Tolterodine or a pharmaceutical acceptable salt, derivative, prodrug and metabolite thereof as an active ingredient due to its useful therapeutical properties. However, the prior art has encountered substantial difficulties in the production of the oral solid formulations of a desirable dissolution profile and a cost effective manufacturing process.

EP 1 128 819 discloses a formulation containing controlled release beads comprising a core unit of a substantially water soluble or water swellable inert material, a first layer on the core of a substantially water insoluble polymer, a second layer over the first that contains an active ingredient and a third layer of polymer on the second layer effective for controlled release of the active ingredient, wherein the first layer is adapted to control water penetration into the core. This process is very complex, not cost effective and time consuming.

WO 2006/21425 discloses a composition comprising a core coated with an outer layer of a hydrophobic sustained release polymer. Said core is either a matrix core made of a matrix core material, Tolterodine and a binder or an inert core being provided with a layer of Tolterodine and a binder.

US 2009/0192228 A1 discloses a controlled-release composition, comprising: inert core comprising a water insoluble polymer; with a first layer disposed on the inert core, wherein the first layer comprises tolterodine and a binder; and a second layer disposed on the first layer, wherein the second layer comprises a water insoluble polymer, a plasticizer, and a pore-forming agent.

WO 2009/080061 A1 discloses a sustained release composition comprising Tolterodine as an active ingredient and a wetting agent such as Sodium Docusate to improve the release of the active ingredient.

All above prior art discloses coated formulations mainly pellets with functional coating with release controlling polymers which provide sustained release of tolterodine, however such formulations requires specialized and complex techniques and are not cost effective.

Although each of the above patents represents an attempt to overcome the problems associated with extended release composition comprising tolterodine however none of them provide extended release composition for tolterodine which is simple, cost effective and remove complicated process of manufacturing, thus there still exists a need to provide a stable, simple, cost effective extended release composition without producing unwanted pharmaceutical side effects and with improved release rate and low production costs.

None of the prior art discloses an extended release composition for tolterodine comprising matrix composition wherein drug release is solely controlled by hydrophobic matrix comprising water insoluble polymer and wax, which is capable of releasing drug for about 24 hours without dose dumping.

### SUMMARY OF THE INVENTION

It is, therefore, an object of the present invention to provide an extended release pharmaceutical composition comprising Tolterodine or pharmaceutical acceptable salts as an active ingredient, which overcomes the deficiencies of the prior art.

In one aspect the present invention provides a stable extended release pharmaceutical composition comprising Tolterodine or pharmaceutical acceptable salts thereof as an active ingredient, which is bioavailable and effective with sufficient shelf-life, good pharmaceutical properties, enhancing patient compliance and reducing possible side effects.

Moreover, yet another aspect of the present invention is to provide an extended release solid pharmaceutical composition comprising Tolterodine or pharmaceutical acceptable salts thereof as an active ingredient, which can be prepared in dosage forms of different strength by proportionally adjusting the quantities of the excipients and the active ingredient, thereby providing a pharmaceutical linearity, without affecting the dissolution profile and bioavailability of the active ingredient.

In a further aspect of the present invention is to provide a method for the preparation of a extended release solid pharmaceutical composition comprising Tolterodine or pharmaceutical acceptable salts thereof as an active ingredient, thereby enhancing the release rate of the active ingredient and being stable over a long period of time and improving the pharmaceutical characteristics of the composition.

### BRIEF DESCRIPTION OF THE DRAWING

FIG. 1 shows dissolution profiles of the pharmaceutical compositions according to the present invention in buffer pH 6.8.

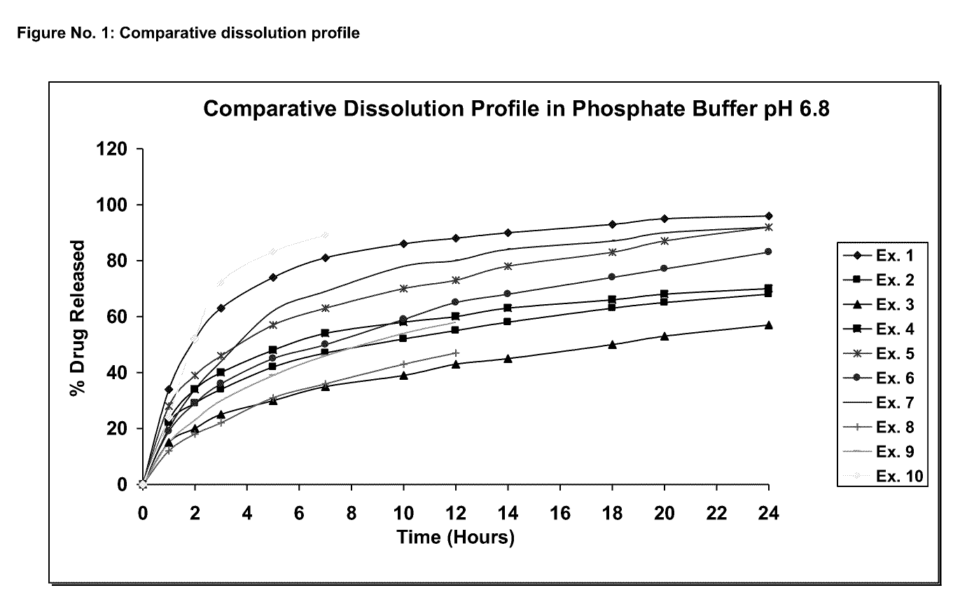

### DETAILED DESCRIPTION OF THE INVENTION

In describing and claiming the present invention, the following terminology will be used in accordance with the definitions set forth below.

The term “tolterodine” herein refers to a compound also commonly known as tolterodine tartrate. The term also refers to analogs and homologs of tolterodine, including salts in addition to the tartrate salt (e.g., citrate, hydrochloride, etc.), prodrugs, enantiomers and metabolites of tolterodine, as well as mixtures thereof, as dictated by the context of its use.

The term “extended release pharmaceutical composition” herein refers to any composition or dosage form that comprises an active drug and which is formulated to provide a longer duration of pharmacological response after administration of the dosage form than is ordinarily experienced after administration of a corresponding immediate release composition comprising the same drug in the same amount. Controlled release compositions include, inter alia, those compositions described elsewhere as “controlled release”, “delayed release”, “sustained release”, “prolonged release”, “programmed release”, “time release” and/or “rate controlled” compositions or dosage forms.

The various embodiments of the present invention can be assembled in several different ways.

In accordance with the above objects the present invention provides, an extended release pharmaceutical composition for oral administration comprising Tolterodine or pharmaceutical acceptable salts thereof comprising water insoluble polymer and/or wax.

In yet another embodiment the present invention provides a process of preparing an extended release pharmaceutical composition such as tablets, capsules and sachets, comprising Tolterodine or pharmaceutical acceptable salts thereof and water insoluble polymer and/or wax.

In yet another embodiment the present invention provides an extended release pharmaceutical composition comprising Tolterodine or pharmaceutical acceptable salts thereof incorporated in a hydrophobic matrix comprising water insoluble polymer and wax wherein weight ratio of water insoluble polymer to wax is from about 1:100 to about 100:1, preferably from about 1:50 to about 50:1, most preferably from about 1:20 to 20:1.

In yet another embodiment the present invention provides an extended release pharmaceutical composition comprising Tolterodine or pharmaceutical acceptable salts thereof incorporated in a hydrophobic matrix comprising water insoluble polymer and wax wherein water insoluble polymer is present from about 1% w/w to about 95% w/w of the composition.

In yet another embodiment the present invention provides an extended release pharmaceutical composition comprising Tolterodine or pharmaceutical acceptable salts thereof incorporated in a hydrophobic matrix comprising water insoluble polymer and wax wherein wax is present from about 1% w/w to about 95% w/w of the composition.

In yet another embodiment the present invention provides an extended release tablet comprising Tolterodine or pharmaceutical acceptable salts thereof incorporated in a hydrophobic matrix comprising water insoluble polymer and wax.

In yet another embodiment the present invention provides an extended release non-swellable tablet comprising Tolterodine or pharmaceutical acceptable salts thereof incorporated in a hydrophobic matrix comprising water insoluble polymer and wax and one or more pharmaceutically acceptable excipients wherein drug release is solely controlled by hydrophobic matrix without dose dumping.

In yet another embodiment the present invention provides, an extended release pharmaceutical composition comprising a hard gelatin capsule with a therapeutically effective number of mini tablets, said mini tablets comprising Tolterodine or pharmaceutical acceptable salts thereof, and water insoluble polymer and wax which is suitable for once daily dosing.

In yet another embodiment the present invention provides an extended release pharmaceutical composition comprising a hard gelatin capsule with a therapeutically effective number of mini tablets, said mini tablets comprising: Tolterodine or pharmaceutical acceptable salts thereof incorporated in a hydrophobic matrix comprising ethyl cellulose and wax selected from group consisting of hydrogenated castor oil, hydrogenated vegetable oil, bees wax, carnauba wax, microcrystalline wax, ozocarite, glyceryl monostearate; glyceryl distearate, glycerol monooleate, acetylated monoglycerides, tristearin, tripalmitin, cetyl esters wax, glyceryl palmitostearate, glyceryl behenate, zein, and one or more pharmaceutically acceptable excipients wherein release of tolterodine is controlled by hydrophobic matrix wherein weight ratio of ethyl cellulose to wax is from about 1:100 to about 100:1, preferably 1:50, to about 50:1, most preferably about 1:20 to 20:1.

In yet another embodiment the present invention provides an extended release uncoated hydrophobic matrix tablet comprising
a) about 0.5% to about 50% by weight of Tolterodine or salt thereof
b) about 1% to about 95% by weight of ethyl cellulose
c) about 1% to about 95% by weight of wax selected from group consisting of hydrogenated castor oil, hydrogenated vegetable oil, bees wax, carnauba wax, microcrystalline wax, ozocarite, glyceryl monostearate; glyceryl distearate, glycerol monooleate, acetylated monoglycerides, tristearin, tripalmitin, cetyl esters wax, glyceryl palmitostearate, glyceryl behenate, zein,
   and one or more pharmaceutically acceptable excipients wherein release of tolterodine is controlled by hydrophobic matrix.

Preferred extended release pharmaceutical compositions according to the present invention comprise Tolterodine or salt thereof in an amount of 0.5% to 50%, more preferably 0.5% to 25% and most preferably 0.5% to 15%.

In yet another embodiment the present invention provides a process of preparing an extended release pharmaceutical composition comprising Tolterodine or pharmaceutical acceptable salts thereof and water insoluble polymer and wax wherein process can be selected from direct compression, dry granulation, wet granulation (aqueous/non-aqueous or combination) and melt granulation.

The present invention can be applied in the formulation of tablets, capsules, caplets, sachets or other solid dosage forms for oral administration of an active ingredient.

In the present invention, an extended release composition in the form of a hard gelatin capsule filled with mini-tablets is provided. The use of mini-tablets is beneficial because pharmaceutical linearity between the strength and the formulation is achieved easily by incorporating one or more of the mini-tablets in the capsule. Linearity is highly desired in the pharmaceutical industry for manufacturing, pharmacokinetics and economical reasons.

Tabletting was the chosen production method because it is faster, easier, adds fewer steps to the process and is the most economical. Further, the tabletting method ensures a high production yield, contrary to the manufacture of pellets where the loss of production output is usually much higher. Excipients for the formulation were chosen carefully to give appropriate dissolution rate and stability of the finished dosage form.

The water insoluble polymers according to present invention includes but are not limited to ethyl cellulose, cellulose acetate, cellulose acetate phthalate, polymethylacrylates, calcium silicates and combinations thereof and the like.

The waxes according to present invention include but are not limited to hydrogenated castor oil, hydrogenated vegetable oil, bees wax, carnauba wax, microcrystalline wax, ozocarite, fatty acid esters such as glyceryl monostearate; glycerol monooleate, acetylated monoglycerides, tristearin, tripalmitin, cetyl esters wax, glyceryl palmitostearate, glyceryl distearate, glyceryl behenate, zein, and combination thereof and the like.

The compositions of the present invention can also include other materials such as binders, diluents, anti-adherents, glidants and lubricants.

Binders may be, for example, starch, sugars, gums, low molecular weight hydroxypropyl methylcellulose, povidone, hydroxypropyl cellulose, hydroxyethyl cellulose or the like.

Diluents may be, for example, any pharmaceutically acceptable, non-toxic diluent. Particular examples include lactose, dextrose, sucrose, maltose, microcrystalline cellulose, starch, calcium hydrogen phosphate, mannitol and the like.

Anti-adherents may be, for example, silicon-containing compounds such as silicon dioxide, magnesium trisilicate, talc and the like.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and the like.

Lubricants may be, for example, talc, magnesium stearate, calcium stearate, stearic acid, sodium stearyl fumarate, sodium benzoate or the like. Antiadherents and Glidants may be, for example, colloidal silicon dioxide, talc or the like.

Solid oral dosage forms of the present invention may be prepared by any conventional techniques for example dry granulation, direct compression, wet granulation, melt granulation and extrusion-spheronization.

The following examples illustrate preferred embodiments in accordance with the present invention without limiting the scope or spirit of the invention.

### Example 1

B. No. 047/003

| | |
|---|---|
| Ingredients | Weight (mg) |

Intra-granular

| | |
|---|---|
| Tolterodine Tartrate | 4.0 |
| Lactose Monohydrate | 60.0 |
| Microcrystalline Cellulose | 80.0 |
| Ethyl Cellulose | 10.0 |
| Alcohol | q.s. |

Extra-granular

| | |
|---|---|
| Hydrogenated Castor Oil | 25.0 |
| Ethyl Cellulose | 20.0 |
| Magnesium Stearate | 1.0 |
| Total | 200.0 |

### Manufacturing Procedure:

1. Sift Tolterodine Tartrate, Lactose monohydrate through suitable sieve.
2. Geometrically mix tolterodine tartrate with lactose monohydrate.
3. Sift Microcrystalline Cellulose and ethyl cellulose through suitable sieve.
4. Mix Step 3 with step 2
5. Granulate step 4 blend by using sufficient quantity of Alcohol.
6. Dry the granules in oven at temperature NMT 50° C.
7. Pass the dried granules through a suitable sieve.
8. Sift Hydrogenated castor oil and ethyl cellulose through suitable sieve.
9. Mix step 7 granules with step 8
10. Sift magnesium Stearate through suitable sieve and lubricate the step 9 blend.
11. Compress the step 10 lubricated blend into tablets/minitablets using suitable punches and/or fill the blend/minitablets into capsules.

### Example 2

B. No. 047/007

| | |
|---|---|
| Ingredients | Weight (mg) |

Intra-granular

| | |
|---|---|
| Tolterodine Tartrate | 4.0 |
| Lactose Monohydrate | 455.0 |
| Ethyl Cellulose | 64.0 |
| Alcohol | q.s. |

Extra-granular

| | |
|---|---|
| Hydrogenated Castor Oil | 80.0 |
| Ethyl Cellulose | 32.0 |
| Magnesium Stearate | 5.0 |
| Total | 640.0 |

### Manufacturing Procedure:

1. Sift Tolterodine Tartrate, Lactose monohydrate through suitable sieve.
2. Geometrically mix tolterodine tartrate with lactose monohydrate.
3. Sift Ethyl Cellulose through suitable sieve.
4. Mix Step 3 with step 2
5. Granulate step 4 blend by using sufficient quantity of Alcohol.
6. Dry the granules in oven at temperature NMT 50° C.
7. Pass the dried granules through a suitable sieve.
8. Sift Hydrogenated castor oil and ethyl cellulose through suitable sieve.
9. Mix step 7 granules with step 8
10. Sift magnesium stearate through suitable sieve and lubricate the step 9 blend.
11. Compress the step 10 lubricated blend into tablets/minitablets using suitable punches and/or fill the blend/minitablets into capsules.

### Example 3

B. No. 047/011

| | |
|---|---|
| Ingredients | Weight (mg) |

Intra-granular

| | |
|---|---|
| Tolterodine Tartrate | 4.0 |
| Lactose Monohydrate | 487.0 |
| Ethyl Cellulose | 32.0 |
| Alcohol | q.s. |

Extra-granular

| | |
|---|---|
| Hydrogenated Castor Oil | 80.0 |
| Ethyl Cellulose | 32.0 |
| Magnesium Stearate | 5.0 |
| Total | 640.0 |

### Manufacturing Procedure:

1. Sift Tolterodine Tartrate, Lactose monohydrate through suitable sieve.
2. Geometrically mix tolterodine tartrate with lactose monohydrate.
3. Sift Ethyl Cellulose through suitable sieve.
4. Mix Step 3 with step 2
5. Granulate step 4 blend by using sufficient quantity of Alcohol.
6. Dry the granules in oven at temperature NMT 50° C.
7. Pass the dried granules through a suitable sieve.
8. Sift Hydrogenated castor oil and ethyl cellulose through suitable sieve.
9. Mix step 7 granules with step 8
10. Sift magnesium stearate through suitable sieve and lubricate the step 9 blend.
11. Compress the step 10 lubricated blend into tablets/minitablets using suitable punches and/or fill the blend/minitablets into capsules.

### Example 4

B. No. 047/019

| | |
|---|---|
| Ingredients | Weight (mg) |

Intra-granular

| | |
|---|---|
| Tolterodine Tartrate | 4.0 |
| Lactose Monohydrate | 122.0 |
| Microcrystalline Cellulose | 50.0 |
| Ethyl Cellulose | 12.5 |
| Alcohol | q.s. |

Extra-granular

| | |
|---|---|
| Hydrogenated Castor Oil | 25.0 |
| Ethyl Cellulose | 25.0 |
| Magnesium Stearate | 1.5 |
| Total | 240.0 |

### Manufacturing Procedure:

1. Sift Tolterodine Tartrate, Lactose monohydrate through suitable sieve.
2. Geometrically mix tolterodine tartrate with lactose monohydrate.
3. Sift Ethyl Cellulose and microcrystalline cellulose through suitable sieve.
4. Mix Step 3 with step 2
5. Granulate step 4 blend by using sufficient quantity of Alcohol.
6. Dry the granules in oven at temperature NMT 50° C.
7. Pass the dried granules through a suitable sieve.
8. Sift Hydrogenated castor oil and ethyl cellulose through suitable sieve.
9. Mix step 7 granules with step 8
10. Sift magnesium stearate through suitable sieve and lubricate the step 9 blend.
11. Compress the step 10 lubricated blend into tablets/minitablets using suitable punches and/or fill the blend/minitablets into capsules.

### Example 5

B. No. 047/027

| | |
|---|---|
| Ingredients | Weight (mg) |

Intra-granular

| | |
|---|---|
| Tolterodine Tartrate | 4.0 |
| Lactose Monohydrate | 81.0 |
| Microcrystalline Cellulose | 108.0 |
| Ethyl Cellulose | 10.0 |
| Ethyl Cellulose (aqueous | 15.0 |
| dispersion) | |
| Purified Water | q.s. |

Extra-granular

| | |
|---|---|
| Hydrogenated Castor Oil | 35.0 |
| Ethyl Cellulose | 14.5 |
| Magnesium Stearate | 2.5 |
| Total | 270.0 |

### Manufacturing Procedure:

1. Sift Tolterodine Tartrate, Lactose monohydrate through suitable sieve.
2. Geometrically mix tolterodine tartrate with lactose monohydrate.
3. Sift Ethyl Cellulose and microcrystalline cellulose through suitable sieve.
4. Mix Step 3 with step 2.
5. Granulate step 4 blend by using ethyl cellulose aqueous dispersion and purified water.
6. Dry the granules in oven at temperature NMT 50° C.
7. Pass the dried granules through a suitable sieve.
8. Sift Hydrogenated Castor Oil and Ethyl Cellulose through suitable sieve.
9. Mix step 7 granules with step 8.
10. Sift magnesium stearate through suitable sieve and lubricate the step 9 blend.
11. Compress the step 10 lubricated blend into tablets/minitablets using suitable punches and/or fill the blend/minitablets into capsules.

### Example 6

B. No. 047/031

| | |
|---|---|
| Ingredients | Weight (mg) |

Intra-granular

| | |
|---|---|
| Tolterodine Tartrate | 4.0 |
| Lactose Monohydrate | 203.5 |
| Ethyl Cellulose | 10.0 |
| Ethyl Cellulose (aqueous dispersion) | 15.0 |
| Hydrogenated Castor Oil | 35.0 |
| Purified Water | q.s. |

Extra-granular

| | |
|---|---|
| Magnesium Stearate | 2.5 |
| Total | 270.0 |

### Manufacturing Procedure:

1. Sift Tolterodine Tartrate, Lactose monohydrate through suitable sieve.
2. Geometrically mix tolterodine tartrate with lactose monohydrate.
3. Sift Ethyl Cellulose, Hydrogenated Castor Oil through suitable sieve.
4. Mix Step 3 with step 2.
5. Granulate step 4 blend by using ethyl cellulose aqueous dispersion and purified water.
6. Dry the granules in oven at temperature NMT 50° C.
7. Pass the dried granules through a suitable sieve.
8. Sift magnesium stearate through suitable sieve and lubricate the step 7 blend.
9. Compress the step 8 lubricated blend into tablets/minitablets using suitable punches and/or fill the blend/minitablets into capsules.

### Example 7

B. No. 047/045

| | |
|---|---|
| Ingredients | Weight (mg) |

Intra-granular

| | |
|---|---|
| Tolterodine Tartrate | 4.0 |
| Lactose Monohydrate | 218.5 |
| Ethyl Cellulose | 30.0 |
| Ethyl Cellulose (aqueous dispersion) | 15.0 |
| Purified Water | q.s. |

Extra-granular

| | |
|---|---|
| Magnesium Stearate | 2.5 |
| Total | 270.0 |

### Manufacturing Procedure:

1. Geometrically mix tolterodine tartrate with lactose monohydrate.
2. Sift step 1 blend, Ethyl Cellulose, through suitable sieve and mix.
3. Granulate step 3 blend by using ethyl cellulose aqueous dispersion and purified water.
4. Dry the granules in oven at temperature NMT 50° C.
5. Pass the dried granules through a suitable sieve.
6. Sift magnesium stearate through suitable sieve and lubricate the step 5 blend.
7. Compress the step 6 lubricated blend into tablets/minitablets using suitable punches and/or fill the blend/minitablets into capsules.

### Example 8

B. No. 047/131

| | |
|---|---|
| Ingredients | Weight (mg) |

Intra-granular

| | |
|---|---|
| Tolterodine Tartrate | 4.0 |
| Lactose Monohydrate | 186.5 |
| Hydrogenated Vegetable Oil | 30.0 |
| Ethyl Cellulose (aqueous dispersion) | 15.0 |
| Purified Water | q.s. |

Extra-granular

| | |
|---|---|
| Talc | 2.0 |
| Magnesium Stearate | 2.5 |
| Total | 240.0 |

### Manufacturing Procedure:

1. Geometrically mix tolterodine tartrate with lactose monohydrate.
2. Sift step 1 blend, and hydrogenated vegetable oil through suitable sieve and mix.
3. Granulate step 3 blend by using ethyl cellulose aqueous dispersion and purified water.
4. Dry the granules in oven at temperature NMT 50° C.
5. Pass the dried granules through a suitable sieve.
6. Sift talc and magnesium stearate through suitable sieve and lubricate the step 5 blend.
7. Compress the step 6 lubricated blend into tablets/minitablets using suitable punches and/or fill the blend/minitablets into capsules.

### Example 9

B. No. 047/135

| | |
|---|---|
| Ingredients | Weight (mg) |

Intra-granular

| | |
|---|---|
| Tolterodine Tartrate | 4.0 |
| Lactose Monohydrate | 186.5 |
| Precirol ATO 5 | 30.0 |
| Ethyl Cellulose | 15.0 |
| Alcohol | q.s. |

Extra-granular

| | |
|---|---|
| Talc | 2.0 |
| Magnesium Stearate | 2.5 |
| Total | 240.0 |

### Manufacturing Procedure:

1. Geometrically mix tolterodine tartrate with lactose monohydrate.
2. Sift step 1 blend, Ethyl Cellulose and Precirol ATO 5 through suitable sieve and mix.
3. Granulate step 2 blend by using sufficient quantity of alcohol.
4. Dry the granules in oven at temperature NMT 50° C.
5. Pass the dried granules through a suitable sieve.
6. Sift talc and magnesium stearate through suitable sieve and lubricate the step 5 blend.
7. Compress the step 6 lubricated blend into tablets/minitablets using suitable punches and/or fill the blend/minitablets into capsules.

### Example 10

B. No. 073/081

| | |
|---|---|
| Ingredients | Weight (mg) |

Intra-granular

| | |
|---|---|
| Tolterodine Tartrate | 4.0 |
| Lactose Monohydrate | 30.0 |
| Precirol ATO 5 | 30.0 |
| Lactose Monohydrate | 161.5 |
| Xanthan Gum | 5.0 |
| Ethyl Cellulose | 5.0 |
| Purified Water | q.s. |

Extra-granular

| | |
|---|---|
| Talc | 2.0 |
| Magnesium Stearate | 2.5 |
| Total | 240.0 |

### Manufacturing Procedure:

1. Geometrically mix tolterodine tartrate with part of lactose monohydrate.
2. Sift step 1 blend, Precirol ATO 5 and ethylcellulose through suitable sieve and mix.
3. Transfer step 2 blend in glass beaker and heat at temperature of about 60° C. under continuous stirring.
4. Cool the step 3 under stirring to form granules.
5. Pass the step 4 granules through suitable sieve and mix with remaining quantity of lactose monohydrate and xanthan gum.
6. Granulate step 5 by using sufficient quantity of purified water.
7. Dry the granules in oven at temperature NMT 50° C.
8. Pass the dried granules through a suitable sieve.
9. Sift talc and magnesium stearate through suitable sieve and lubricate the step 8 blend.
10. Compress the step 9 lubricated blend into tablets/minitablets using suitable punches and/or fill the blend/minitablets into capsules.

The formulations of Example 1 to Example 10 were subjected to in-vitro dissolution studies and the results obtained are presented below table:

| Dissolution Studies | Dissolution Media: 900 ml, pH 6.8 Phosphate Buffer, USP I, 100 RPM | | | | | | | | | |
|---|---|---|---|---|---|---|---|---|---|---|
| Time (hrs) | % Drug Released | | | | | | | | | |
| Exam. | Ex. 1 | Ex. 2 | Ex. 3 | Ex. 4 | Ex. 5 | Ex. 6 | Ex. 7 | Ex. 8 | Ex. 9 | Ex. 10 |
| 1 | 34 | 22 | 15 | 23 | 28 | 19 | 20 | 12 | 15 | 24 |
| 2 | 52 | 29 | 20 | 34 | 39 | 29 | 34 | 18 | 23 | 52 |
| 3 | 63 | 34 | 25 | 40 | 46 | 36 | 44 | 22 | 30 | 72 |
| 5 | 74 | 42 | 30 | 48 | 57 | 45 | 62 | 31 | 39 | 83 |
| 7 | 81 | 47 | 35 | 54 | 63 | 50 | 69 | 36 | 46 | 89 |
| 10 | 86 | 52 | 39 | 58 | 70 | 59 | 78 | 43 | 54 | — |
| 12 | 88 | 55 | 43 | 60 | 73 | 65 | 80 | 47 | 58 | — |
| 14 | 90 | 58 | 45 | 63 | 78 | 68 | 84 | — | — | — |
| 18 | 93 | 63 | 50 | 66 | 83 | 74 | 87 | — | — | — |
| 20 | 95 | 65 | 53 | 68 | 87 | 77 | 90 | — | — | — |
| 24 | 96 | 68 | 57 | 70 | 92 | 83 | 92 | — | — | — |

We claim:

1. An extended release pharmaceutical composition comprising a hard gelatin capsule with a therapeutically effective number of mini tablets, said mini tablets comprising: Tolterodine or pharmaceutical acceptable salts thereof incorporated in a hydrophobic matrix comprising water insoluble polymer and wax and one or more pharmaceutically acceptable excipients wherein release of tolterodine is controlled by hydrophobic matrix.

2. An extended release pharmaceutical composition according to claim 1 wherein water insoluble polymer is selected from group consisting of ethyl cellulose, cellulose acetate, cellulose acetate phthalate, polymethyacrylates, calcium silicates.

3. An extended release pharmaceutical composition according to claim 1 wherein wax is selected from group consisting of hydrogenated castor oil, hydrogenated vegetable oil, bees wax, carnauba wax, microcrystalline wax, ozocarite, glyceryl monostearate; glyceryl distearate, glycerol monooleate, acetylated monoglycerides, tristearin, tripalmitin, cetyl esters wax, glyceryl palmitostearate, glyceryl behenate, zein.

4. An extended release pharmaceutical composition according to claim 1 wherein weight ratio of water insoluble polymer to wax is from about 1:100 to about 100:1.

5. An extended release pharmaceutical composition according to claim 1 wherein weight ratio of water insoluble polymer to wax is from about 1:50 to about 50:1.

6. An extended release pharmaceutical composition according to claim 1 wherein weight ratio of water insoluble polymer to wax is from about 1:20 to about 20:1.

7. An extended release pharmaceutical composition according to claim 1 wherein water insoluble polymer is present from about 1% w/w to about 95% w/w of the composition.

8. An extended release pharmaceutical composition according to claim 1 wherein wax is present from about 1% w/w to about 95% w/w of the composition.

9. An extended release pharmaceutical composition according to claim 1 wherein the tablet is prepared using direct compression, dry granulation, wet granulation or melt granulation.

10. An extended release uncoated hydrophobic matrix tablet comprising
a) about 0.5% to about 50% by weight of Tolterodine or salt thereof
b) about 1% to about 95% by weight of ethyl cellulose
c) about 1% to about 95% by weight of wax which is selected from group consisting of hydrogenated castor oil, hydrogenated vegetable oil, bees wax, carnauba wax, microcrystalline wax, ozocarite, glyceryl monostearate; glyceryl distearate, glycerol monooleate, acetylated monoglycerides, tristearin, tripalmitin, cetyl esters wax, glyceryl palmitostearate, glyceryl behenate, zein, and one or more pharmaceutically acceptable excipients wherein release of tolterodine is controlled by hydrophobic matrix.

11. An extended release pharmaceutical composition according to claim 10 wherein weight ratio of ethyl cellulose to wax is from about 1:100 to about 100:1.

12. An extended release pharmaceutical composition according to claim 10 wherein weight ratio of ethyl cellulose to wax is from about 1:50 to about 50:1.

13. An extended release pharmaceutical composition according to claim 10 wherein weight ratio of ethyl cellulose to wax is from about 1:20 to about 20:1.

14. An extended release pharmaceutical composition according to claim 10 wherein the tablet is prepared using direct compression, dry granulation, wet granulation or melt granulation.
